(19) United States
(12) Patent Application Publication
(10) Pub. No.: US 2009/0215851 A1
(43) Pub. Date: Aug. 27, 2009

(54) Method of Preparing a Porphyrin Derivative, a Porphyrin Derivative, Use of Said Porphyrin derivative and a pharmaceutical composition containing said porphyrin derivative

(75) Inventors: Hendrikus, Nicolaas, Sebastiaan Van Der Haas, Nijmegen (NL); Johannis Lugtenburg, Oegstgeest (NL); Johannes Schuitmaker, Leiden (NL); Robertus Leendert Pieter De Jong, Sassenheim (NL)

Correspondence Address: HOWREY LLP-EU, C/O IP DOCKETING DEPARTMENT, 2941 FAIRVIEW PARK DR., SUITE 200, FALLS CHURCH, VA, 22042, US

(73) Assignee: PHOTOBIOCHEM N.V., Leiden (NL)

(21) Appl. No.: 11/885,848

(22) PCT Filed: Mar. 8, 2006
(86) PCT No.: PCT/NL06/00119

§ 371 Date: Feb. 23, 2009

(30) Foreign Application Priority Data
Mar. 8, 2005 (NL) 1028485

(51) Int. Cl. A61K 31/409; C07D 487/22; A61P 17/00; A61P 35/00; A61P 27/02; A61P 37/00; A01N 43/38; A01P 7/00; A01P 1/00

(52) U.S. Cl. 514/410; 540/145

(57) ABSTRACT

The present invention relates to a method of preparing a porphyrin derivative from a meso-acrylonitrile-substituted porphyrin compound. According to the invention a Vilsmeier reagent carrying an aromatic ring is used, wherein said Vilsmeier reagent is a soft electrophile. The use of this Vilsmeier reagent results in a porphyrin derivative having the ring system of the Vilsmeier reagent condensed to its porphyrin macrocycle via two new 6 membered rings. Thus a new class of porphyrin derivatives is provided, wherein said derivatives are photodynamically more active than the starting compound. The invention also relates to a porphyrin derivative, use of said porphyrin derivative and a pharmaceutical composition containing said porphyrin derivative.

The present invention relates to a method of preparing a porphyrin derivative from a meso-acrylonitrile-substituted porphyrin compound, wherein said meso-acrylonitrile-substituted porphyrin compound contains a coordinated bivalent metal ion in the porphyrin macrocycle.

Such a method is known in the art. In particular, Van der Haas et al describe (Eur. J. Org. Chem. (2004), 19, pp. 4024-4038) a method of preparing a porphyrin derivative having a quinoline ring system peri-condensed to the porphyrin macrocycle. The method results in a porphyrin derivative having a favourable property, more specifically a stronger absorption shifted towards the far red compared to that of the meso-acrylonitrile-substituted starting compound. This longer absorption wavelength makes the porphyrin derivative prepared using this new method in particular suitable for in vivo photodynamic therapy applications where light has to penetrate tissue deeply.

In the art, there is a continuous need for methods that result in classes of porphyrin derivatives having an improved characteristic.

Accordingly, the object of the method according to the present invention is to provide a new method for preparing a new class of porphyrin derivatives, wherein said porphyrin derivates are essentially non-toxic in the dark and in the presence of oxygen while showing excellent photodynamic activity (such as toxicity to cells, generated in the presence of oxygen and light of a wavelength that is absorbed by the porphyrin derivative).

Thus the present invention provides a method which is characterized in that the meso-acrylonitrile-substituted porphyrin compound, in a form wherein said meso-acrylonitrile-substituted porphyrin compound is complexed with a bivalent metal ion, is contacted with a Vilsmeier reagent having a reactive motif

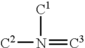

containing a quaternary nitrogen atom which is directly linked to two carbon atoms C¹, C² wherein at least one of said carbon atoms is part of an aromatic moiety, and wherein said quaternary nitrogen atom is directly linked to a carbon atom C³ via a double bond, said carbon atom C³ carrying a halogen atom chosen from fluoro, chloro, bromo and iodo,

with the restriction that at least one C atom of the aromatic moiety ortho with respect to the quaternary nitrogen is unsubstituted;

to convert said meso-acrylonitrile-substituted porphyrin compound into a porphyrin derivative having the ring system of the Vilsmeier reagent condensed to its porphyrin macrocycle via two new 6 membered rings.

This leads, surprisingly, to a new class of compounds having the ring system of the Vilsmeier reagent used, and, concommitantly, display improved photodynamic activity in comparison to the starting compounds. Other advantages are that the new compounds are very stable, and moreover, compared to their starting compounds, they have a higher absorption coefficient, and the absorption maximum shifts towards the red, which is quite desirable for in vivo photodynamic purposes.

Even though the ring system of the Vilsmeier reagent is not linked via double bonds or via an aromatic ring to the porphyrin macrocycle, it still influences the wavelength of the absorption peak, which allows the person skilled in the art to prepare and select a porphyrin derivative suitable for his/her particular needs. The Vilsmeier reagent as defined above can be designated as a soft electrophile, whose soft nature is, without being bound to any particular theory, believed to be essential for obtaining this new class of compounds. The reaction is conveniently performed at room temperature, but may be performed at higher or lower temperatures as desired. Optionally, selecting or modifying substituents on the ring system derived from the Vilsmeier reagent allow further fine tuning of the properties desired for a particular purpose, increasing the usefulness of the method according to the present invention. Preferably the coordinated bivalent metal ion is nickel in the method according to the invention. In the present application the term meso-acrylonitrile indicates optionally substituted meso-(2′-cyanovinyl). If a porphyrin starting compound doesn't contain a meso-acrylonitrile group, it can readily be introduced according to any method known in the art, such as disclosed by Van der Haas (supra).

Preferably, a Vilsmeier reagent is used in which the halogen atom is chloro. Such a Vilsmeier reagent facilitates obtaining the porphyrin derivative having improved photodynamic activity.

Another preferred embodiment is characterized in that a Vilsmeier reagent is used chosen from the group consisting of

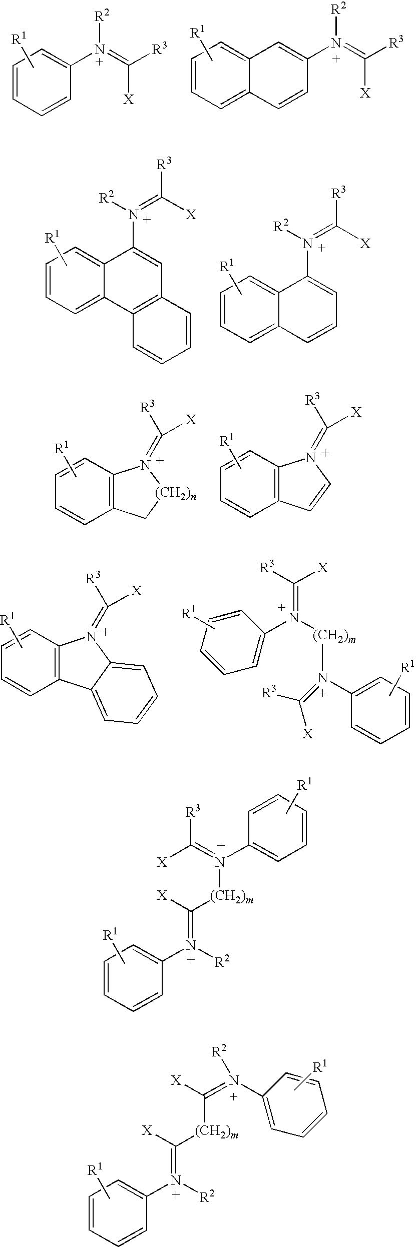

wherein

n is an integer from 0 to 3

m is an integer from 1 to 8

X is an halogen atom chosen from fluoro, chloro, bromo, and iodo;

R¹ is
hydrogen, or
has the same meaning as defined for R², in which case it may be condensed with an aromatic ring of the ring system of the Vilsmeier reagent, whose aromatic ring carries the quaternary nitrogen atom;

R² is
a saturated or unsaturated C₁₋₆ alkyl residue, wherein said alkyl residue may optionally be substituted with one or more substituents independently chosen from the group of linear or branched C₁₋₆ alkoxy, linear or branched C₁₋₆ alkylthio, linear or branched C₂₋₆ alkenyl, linear or branched C₂₋₆ alkynyl, chloro, bromo or fluoro, carbonyl, C₆₋₁₂ aryl, or amino substituted with two substituents independently chosen from the group of linear or branched C₁₋₆ alkoxy, linear or branched C₁₋₆ alkylthio, and C₆₋₁₂ aryl where these substituents of the amine group may optionally be substituted with fluoro, chloro, bromo, and iodo; wherein the saturated or unsaturated C₁₋₆ alkyl residue may be part of a ring system condensed with the aromatic ring carrying the nitrogen atom of the Vilsmeier reagent;

or
a C₆₋₁₂ aryl residue, wherein said aryl residue may optionally be substituted with one or more substituents independently chosen from the group of linear or branched C₁₋₆ alkyl, linear or branched C₁₋₆ alkoxy, linear or branched C₁₋₆ alkylthio, linear or branched C₂₋₆ alkenyl, linear or branched C₂₋₆ alkynyl, chloro, bromo or fluoro, carbonyl, or amino substituted with two substituents independently chosen from the group of linear or branched C₁₋₆ alkoxy, linear or branched C₁₋₆ alkylthio, and C₆₋₁₂ aryl where these substituents of the amine group may optionally be substituted with fluoro, chloro, bromo, and iodo; wherein the C₆₋₁₂ aryl residue may be part of a ring system condensed with the aromatic ring carrying the nitrogen atom of the Vilsmeier reagent; and

R³ is hydrogen or is as defined for R².

Preferably the halogen atom of the Vilsmeier reagent is chloro.

Yet another preferred embodiment is characterized in that a meso-acrylonitrile-substituted porphyrin compound of the formula (I) is used as a starting compound

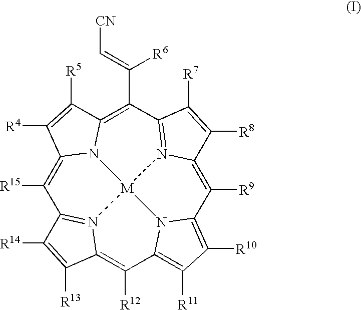

wherein

R⁴, R⁵, R⁷ and R⁸ represent, independently of each other, hydrogen, linear or branched (C₁₋₈) alkyl, or linear or branched (C₁₋₈)alkyl C(O)O(C₁₋₈)alkyl, wherein the alkyl groups may optionally be substituted with fluoro, chloro, bromo, iodo, nitrile, (C₁₋₈) thioether, and (C₁₋₈) alkoxy;

R⁶ represents hydrogen, nitrile, monocyclic, bicyclic or tricyclic (C₆₋₁₄) aryl, or (C₁₋₄) alkyl wherein the aryl and alkyl group may optionally be substituted with fluoro, chloro, bromo, iodo, nitrile, (C₁₋₈) thioether, and (C₁₋₈) alkoxy, and the alkyl group may be substituted with a monocyclic, bicyclic or tricyclic (C₆₋₁₄) aryl;

R⁹ to R¹⁵ represent independently of each other, hydrogen, linear or branched (C₁₋₈) alkyl, linear or branched (C₁₋₈)alkyl C(O)O(C₁₋₈)alkyl, wherein n is an integer of 0 to 4, CH₂═CH—, a monocyclic, bicyclic or tricyclic (C₃-C₁₄) aryl, said aryl optionally containing one or more nitrogen atoms as heteroatoms; and R⁹, R¹², and R¹⁵ may in addition represent an acrylonitrile group substituted with R⁶′₁, wherein R⁶′ is as defined for R⁶;

and

M represents a bivalent metal ion or two hydrogen atoms.

According to a favourable embodiment, the porphyrin derivative is formylated.

Thus the method allows for the preparation of compounds which can easily be coupled, using methods well known in the art, to a variety of substrates and molecules. To achieve the formylation, the method merely requires an excess of the Vilsmeier reagent followed by subjecting the reaction product to hydrolysis. Other possibilities afforded by the formylation is attachment to surfaces, changing of pharmacokinetical properties, water solubility, etc.

To change a property of the porphyrin derivative and make it more suitable for the intended purpose, it may be advantageous if the bivalent metal ion is removed or replaced by another metal ion.

To perform a reaction according to the invention, the use of Ni²⁺ as the bivalent metal ion was shown to result in good yields. The Ni², or any other bivalent metal ion used may be removed (by introducing two hydrogen atoms) or replaced after reaction, using methods well known in the art (References: Fuhrop, J. H. et al in Porphyrins and Metalloporphyrins, Smith, K. M.; Ed Elsevier: Amsterdam, 1975; p. 185 and pp. 795-798. Buchler, J. W.; In The Porphyrins; Dolphin, D.; Ed.; Academic Press, New York 1978, Vol 1A, p. 389. Sanders et al.; In The Porphyrin Handbook, Kadish, K. M., 20 Smith, K. M. and Guilard, R.; Ed.; Academic Press, San Diego, 2000, Vol 3, Chapter 15, pp. 3-40. demetallation of porphyrins: Fuhrop, J. H. et al in Porphyrins and Metalloporphyrins, Smith, K. M.; Ed Elsevier: Amsterdam, 1975; pp. 195-207 and pp. 243-247. Particular reference is made to Buchler, J. W. et 25 al, Liebigs annalen der Chemie (1988), pp. 43-54). For example, for therapeutic purposes, the metal in the porphyrin derivative, if present should not quench the excited triplet state of the porphyrin. To this end, nickel may be replaced by another metal ion, for example a metal ion of reduced toxicity such as zinc or palladium. Other reasons to remove or replace a metal ion are, for example, to change the porphyrin derivative's characteristics, for example, optical characteristics or photodynamic activity.

According to a favourable embodiment, the nitrogen atom of the newly formed ring system is quaternized.

This may be achieved using methods well known in the art, such as alkylation. Thus the method according to the invention not only allows the preparation of porphyrin derivatives having improved photodynamic activity, but provides in addition the possibility to tune the wavelength, in particular towards the red end of the spectrum and to improve the solubility in aqueous solutions, such as blood or saline, or in an alcohol-based pharmaceutical excipient.

According to a preferred embodiment, the meso-acrylonitrile-substituted porphyrin compound is derived from a precursor porphyrin compound chosen from the group of i) hemin, and ii) heme.

These precursors for preparing the starting compounds to be used in the method according to the invention are cheap, allowing a more economical preparation of the compounds according to the invention.

The present invention also relates to a porphyrin derivative obtainable with the method according to the invention, its enantiomers, as well as the addition salts thereof with an acid or base. Specific examples of favourable compounds are
2′-Cyano-8′-formyl-N-methyl-1′,1a′,5a′,6′-tetrahydroacrido[4,5,5a,6-bcd]-annulated 2,3-dihydromesoporphyrin dimethylester;
2′-Cyano-8′-formyl-N-methyl-1′,1a′,5a′,6′-tetrahydroacrido[4,5,5a,6-abt]-annulated 2,3-dihydromesoporphyrin dimethylester;
2′-Cyano-8′-formyl-N-methyl-1′,1a′,5a′,6′-tetrahydroacrido[4,5,5a,6-efg]-annulated 7,8-dihydromesoporphyrin dimethylester;
2′-Cyano-8′-formyl-N-methyl-1′,1a′,5a′,6′-tetrahydroacrido[4,5,5a,6-ghi]-annulated 7,8-dihydromesoporphyrin dimethylester;
2′-Cyano-8′-formyl-N-methyl-1′,1a′,5a′,6′-tetrahydroacrido[4,5,5a,6-jkl]-annulated 12,13-dihydromesoporphyrin dimethylester;
2′-Cyano-8′-formyl-N-methyl-1′,1a′,5a′,6′-tetrahydroacrido[4,5,5a,6-lmn]-annulated 12,13-dihydromesoporphyrin dimethylester;
2′-Cyano-8′-formyl-N-methyl-1′,1a′,5a′,6′-tetrahydroacrido[4,5,5a,6-opq]-annulated 17,18-dihydromesoporphyrin dimethylester;
2′-Cyano-8′-formyl-N-methyl-1′,1a′,5a′,6′-tetrahydroacrido[4,5,5a,6-qrs]-annulated 17,18-dihydromesoporphyrin dimethylester;
2′-Cyano-8′-formyl-N-methyl-1′,1a′,5a′,6′-tetrahydroacrido[4,5,5a,6-bcd]-annulated 2,3-dihydromesoporphyrin;
2′-Cyano-8′-formyl-N-methyl-1′,1a′,5a′,6′-tetrahydroacrido[4,5,5a,6-abt]-annulated 2,3-dihydromesoporphyrin;
2′-Cyano-8′-formyl-N-methyl-1′,1a′,5a′,6′-tetrahydroacrido[4,5,5a,6-efg]-annulated 7,8-dihydromesoporphyrin;
2′-Cyano-8′-formyl-N-methyl-1′,1a′,5a′,6′-tetrahydroacrido[4,5,5a,6-ghi]-annulated 7,8-dihydromesoporphyrin;
2′-Cyano-8′-formyl-N-methyl-1′,1a′,5a′,6′-tetrahydroacrido[4,5,5a,6-jkl]-annulated 12,13-dihydromesoporphyrin;
2′-Cyano-8′-formyl-N-methyl-1′,1a′,5a′,6′-tetrahydroacrido[4,5,5a,6-lmn]-annulated 12,13-dihydromesoporphyrin;
2′-Cyano-8′-formyl-N-methyl-1′,1a′,5a′,6′-tetrahydroacrido[4,5,5a,6-opq]-annulated 17,18-dihydromesoporphyrin;
2′-Cyano-8′-formyl-N-methyl-1′,1a′,5a′,6′-tetrahydroacrido[4,5,5a,6-qrs]-annulated 17,18-dihydromesoporphyrin;
2′-Aminocarbonyl-8′-formyl-N-methyl-1′,1a′,5a′,6′-tetrahydroacrido[4,5,5a,6-bcd]-annulated 2,3-dihydromesoporphyrin dimethylester;
2′-Aminocarbonyl-8′-formyl-N-methyl-1′,1a′,5a′,6′-tetrahydroacrido[4,5,5a,6-abt]-annulated 2,3-dihydromesoporphyrin dimethylester;
2′-Aminocarbonyl-8′-formyl-N-methyl-1′,1a′,5a′,6′-tetrahydroacrido[4,5,5a,6-efg]-annulated 7,8-dihydromesoporphyrin dimethylester;
2′-Aminocarbonyl-8′-formyl-N-methyl-1′,1a′,5a′,6′-tetrahydroacrido[4,5,5a,6-ghi]-annulated 7,8-dihydromesoporphyrin dimethylester;
2′-Aminocarbonyl-8′-formyl-N-methyl-1′,1a′,5a′,6′-tetrahydroacrido[4,5,5a,6-jkl]-annulated 12,13-dihydromesoporphyrin dimethylester;
2′-Aminocarbonyl-8′-formyl-N-methyl-1′,1a′,5a′,6′-tetrahydroacrido[4,5,5a,6-lmn]-annulated 12,13-dihydromesoporphyrin dimethylester;
2′-Aminocarbonyl-8′-formyl-N-methyl-1′,1a′,5a′,6′-tetrahydroacrido[4,5,5a,6-opq]-annulated 17,18-dihydromesoporphyrin dimethylester;
2′-Aminocarbonyl-8′-formyl-N-methyl-1′,1a′,5a′,6′-tetrahydroacrido[4,5,5a,6-qrs]-annulated 17,18-dihydromesoporphyrin dimethylester
2′-Aminocarbonyl-8′-formyl-N-methyl-1′,1a′,5a′,6′-tetrahydroacrido[4,5,5a,6-bcd]-annulated 2,3-dihydromesoporphyrin;
2′-Aminocarbonyl-8′-formyl-N-methyl-1′,1a′,5a′,6′-tetrahydroacrido[4,5,5a,6-abt]-annulated 2,3-dihydromesoporphyrin;
2′-Aminocarbonyl-8′-formyl-N-methyl-1′,1a′,5a′,6′-tetrahydroacrido[4,5,5a,6-efg]-annulated 7,8-dihydromesoporphyrin;
2′-Aminocarbonyl-8′-formyl-N-methyl-1′,1a′,5a′,6′-tetrahydroacrido[4,5,5a,6-ghi]-annulated 7,8-dihydromesoporphyrin;
2′-Aminocarbonyl-8′-formyl-N-methyl-1′,1a′,5a′,6′-tetrahydroacrido[4,5,5a,6-jkl]-annulated 12,13-dihydromesoporphyrin;
2′-Aminocarbonyl-8′-formyl-N-methyl-1′,1a′,5a′,6′-tetrahydroacrido[4,5,5a,6-lmn]-annulated 12,13-dihydromesoporphyrin;
2′-Aminocarbonyl-8′-formyl-N-methyl-1′,1a′,5a′,6′-tetrahydroacrido[4,5,5a,6-opq]-annulated 17,18-dihydromesoporphyrin;
2′-Aminocarbonyl-8′-formyl-N-methyl-1′,1a′,5a′,6′-tetrahydroacrido[4,5,5a,6-qrs]-annulated 17,18-dihydromesoporphyrin;
2′-aminocarbonyl-8′-formyl-N′-methyl-1′,1a′,5a′,6′-tetrahydroacrido[4,5,5a,6-bcd]-annulated 2,3-dihydromesoporphyrin dimethylester;
2′-cyano-8′-formyl-N′-methyl-1′,1a′,5a′,6′-tetrahydroacrido[4,5,5a,6-bcd]-annulated 2,3-dihydromesoporphyrin dicarboxylic acid;
2′-cyano-9′,N′-dimethyl-8′-formyl-1′,1a′,5a′,6′-tetrahydroacrido[4,5,5a,6-bcd]-annulated 2,3-dihydromesoporphyrin dimethylester; and
2′-cyano-8′-formyl-N′-methyl-1′,1a′,5a′,6′-tetrahydroacrido[4,5,5a,6-bcd]-annulated octaethyl porphyrin
   as well as the addition salts thereof with pharmaceutically acceptable acid or base.

The present invention also relates to the use of a porphyrin derivative according to the invention for the preparation of a pharmaceutical composition for treating those benign, malignant, inflamed and infectious indications that are well known in the art for clinical and other uses of photosensitizers. These indications include amongst others:

1) Skin and mucosa disorders, benign, malignant, inflamed and infectious skin/mucosa disorders such as acne, warts, eczema, birthmarks (including vascular malformations such as naevus flammeus and hyperpigmentation), hirsutism skin/(burn)wound/mucosa infections (caused by bacteria, viruses, dermatophytes and other fungi, yeasts and/or parasites), actinic keratoses, psoriasis, primary tumors (including basal cell carcinomas, squamous cell carcinoma and melanomas) and secondary tumors of the skin and mucosa. In addition, photosensitizers can be used to decontaminate the skin and mucosa for the prevention of infections;

2) Vascular disorders: vascular diseases such as the different kinds of macula degeneration in opthalmology, treatment of atherosclerotic plaques, prevention and/or treatment of vascular (re)stenose or aneurysms, arterio-venous malformations and other vascular anomalies;

3) Oncology: as an alternative or addition to the standard treatment of tumors and pre-cancerous lesions such as pancreas head cancer, tumors of the brain, lung, cervix, uterus, urinary bladder, bile bladder, stomach, gut, thyroid and oesophagus (including Barret's oesophagus), prostate cancer, head and neck cancers (including cancers of the oral cavity, ears, nose, larynx and pharynx) and kidney tumors;

4) Opthalmology disorders: disorders in the eye such as age-related macula degeneration, secondary cataract, infections, immunological diseases and tumors;

5) Gynecological or urological disorders: urogenital diseases such as uterus bleedings, endometriosis, benign prostate hypertrophy and for use in endometrial ablation;

6) Immunological disorders: diseases caused by aberrations of the immune system or increased inflammatory reactions such as multiple sclerosis, rheumatoid arthritis, Inflammatory Bowel Disease (including colitis ulcerosa and Crohn's disease), scleroderma and thyroiditis;

7) Oral cavity or nasopharyngeal disorders, including dentistry applications, for example disorders in the oral cavity such as decontamination of root canals, treatment and/or prevention of gum disease and treatment of wounds or other mucosal disorders.

Preferred uses are characterized in that a porphyrin derivative according to the invention is used for the preparation of a pharmaceutical composition for treating a disorder wherein the disorder is a vascular anomaly, and more particular wherein the vascular anomaly is Age-related Macula Degeneration. Other preferred uses are characterized in that a porphyrin derivative according to the invention is used for the preparation of a pharmaceutical composition for treating a disorder wherein the disorder is a primary tumor and/or metastases thereof, and more in particular, wherein the primary tumor and/or metastases thereof is a mammary tumor and/or metastases thereof.

The porphyrin derivative in a pharmaceutical composition according to the invention may be present in any suitable form, including as its acid or basic addition salt or as the free base and free acid thereof and the pharmaceutical composition will generally include a pharmaceutically acceptable carrier or excipient. In general, porphyrins not containing a metal ion will be preferred. With respect to those porphyrin derivatives according to the invention having one or more ester groups, the free acids or addition salts of those porphyrin derivatives are considered to be preferred for pharmaceutical compositions in view of their improved solubility in aqueous solutions, including blood. Saponification of these esters can be accomplished using standard methods known in the art, such as hydrolyzing using sodium hydroxide. Thus, the present invention relates to a pharmaceutical composition comprising a porphyrin derivative according to the invention together with a pharmaceutically acceptable carrier or excipient. More in particular, the pharmaceutical composition is suitable for the treatment of benign, malignant, inflamed and infectious

1) skin and mucosa disorders;

2) vascular disorders;

3) tumors and pre-cancerous lesions;

4) opthalmology disorders;

5) gynaecological or urological disorders;

6) immunological disorders;

7) oral cavity or nasopharyngeal disorders.

According to a preferred embodiment, the pharmaceutical composition is a pharmaceutical composition suitable for the treatment of vascular anomalies, in particular Age-related Macula Degeneration. According to another preferred embodiment, the pharmaceutical composition is a pharmaceutical composition suitable for the treatment of primary tumors and/or the metastases thereof, in particular mammary tumors and/or metastases thereof.

In addition, the derivatives according to the present invention are useful

1) for photodetection of malignant and pre-malignant lesions for instance in the bladder, lung or esophagus;

2) for decontamination or pathogen (such as gram positive and negative bacteria, viruses, parasites, prions and fungi) reduction in liquids such as biological fluids (including donor blood, stem cell containing fluids, bone marrow purging) and contaminated water;

3) for decontamination or pathogen reduction of surfaces either by using liquid photosensitizers or by coupling them directly to the said surface;

4) for use as insecticide.

The appended claims are included in the description by reference.

Finally, the invention relates to a non-pharmaceutical composition comprising a porphyrin derivative according to the invention together with an acceptable carrier or excipient.

The present invention is elucidated with reference to the following non-limiting examples, and with reference to the drawing in which

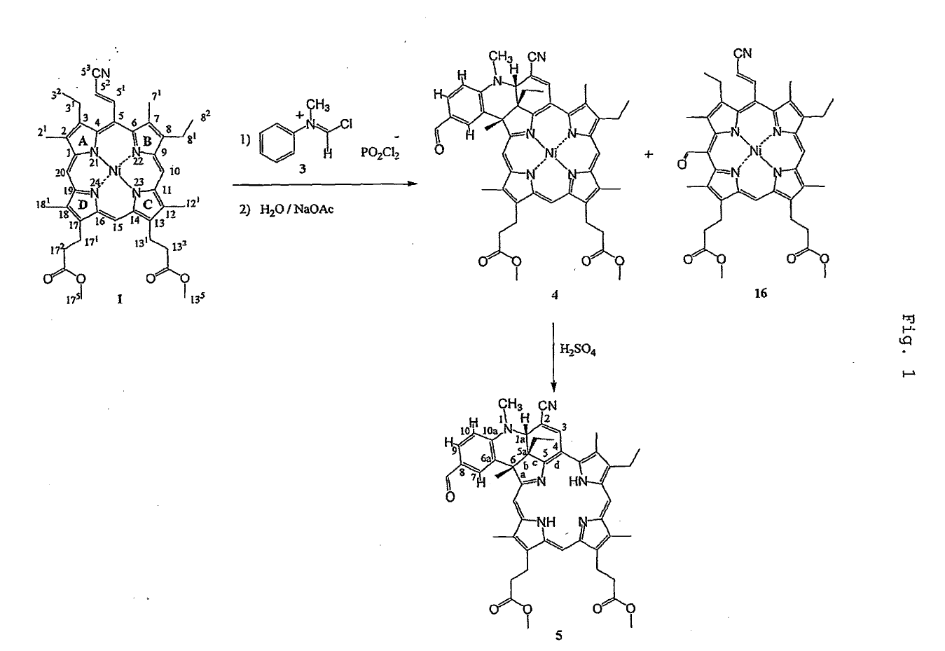

FIG. 1 is a scheme depicting the reaction of an acrylonitrile porphyrin compound and a Vilsmeier reagent using the method according to the invention into a porphyrin derivative having the ring system of the Vilsmeier reagent condensed to its porphyrin macrocycle via two new 6 membered rings (for compound 16, a by-product, only one of its isomers is shown.);

FIG. 2 is a graph displaying the dark toxicity of compound 5 of FIG. 1;

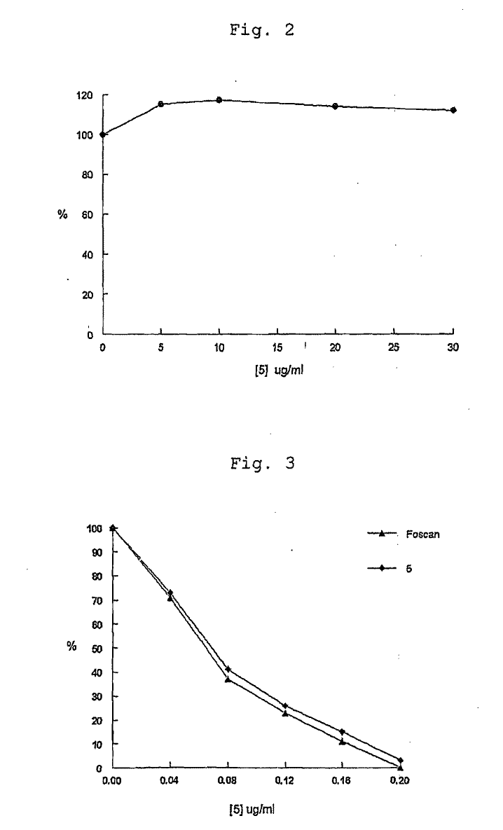

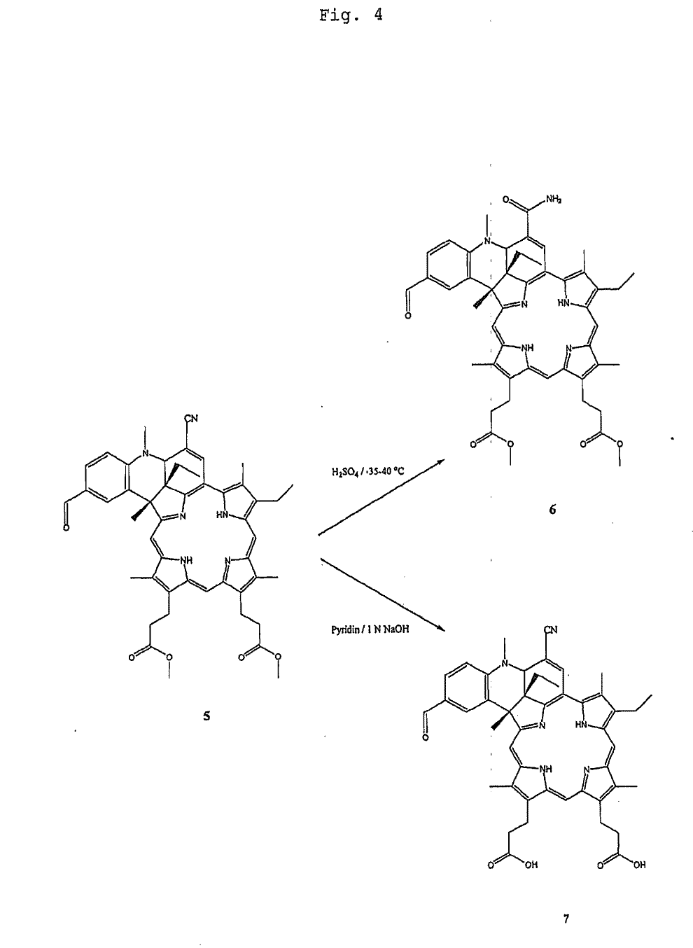

FIG. 3 is a graph comparing the photodynamic cytotoxicity of compound 5 of FIG. 1 with Foscan™ as a reference compound, Foscan™ being a chlorin having the highest photodynamic cytotoxicity known to the inventors;

FIG. 4 displays two compounds, 6 and 7, according to the present invention;

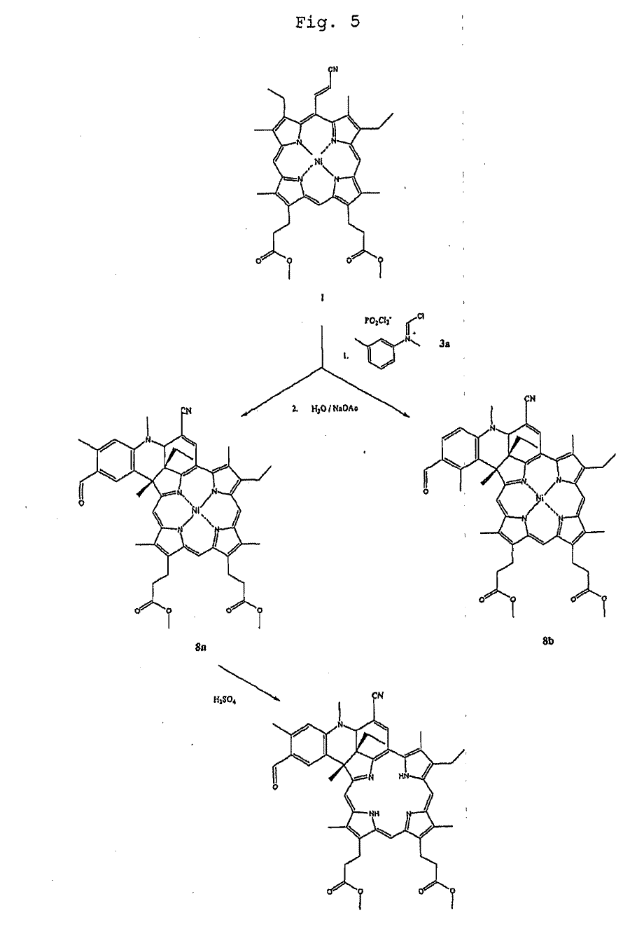

FIG. 5 displays a compound, compound 9, according to the present invention;

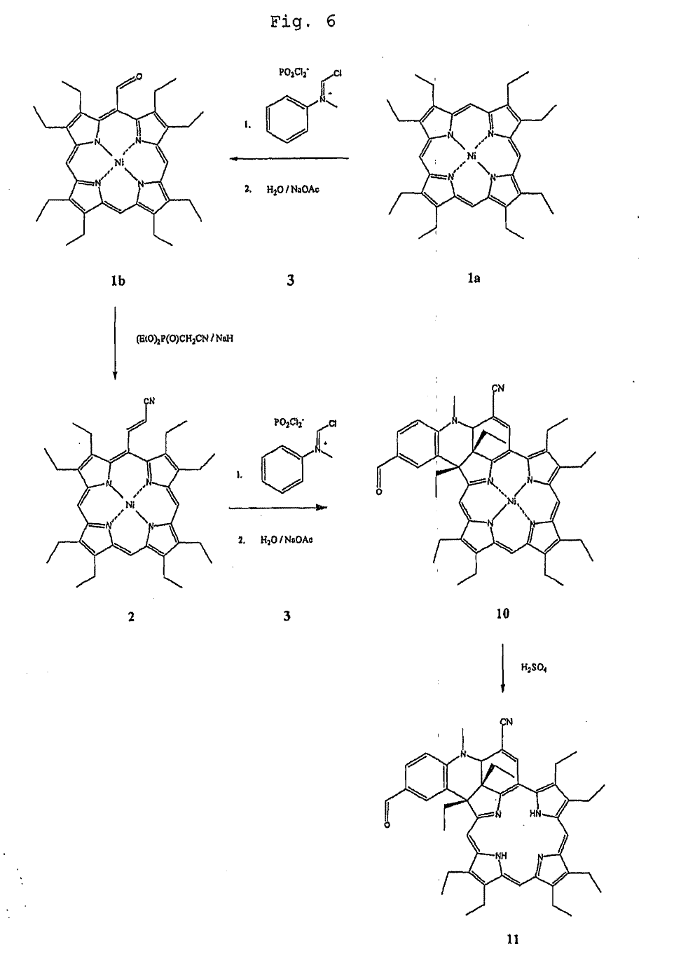

FIG. 6 is a scheme displaying the synthesis of compound 11;

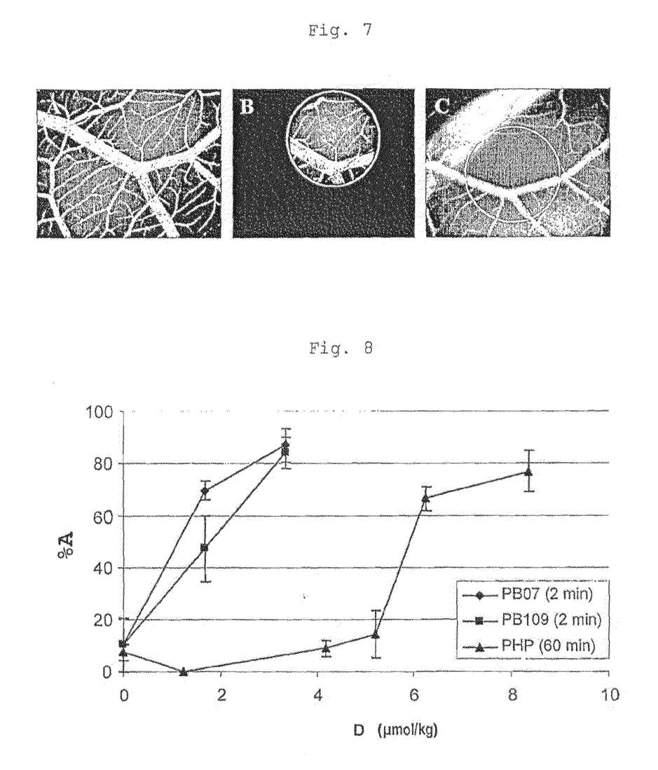

FIG. 7 displays blood vessels, before, during and after photodynamic treatment using compound 7;

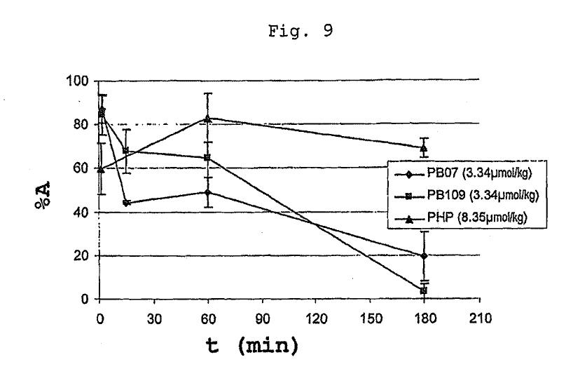

FIGS. 8 and 9 demonstrate the excellent properties of compound 7 in cancer therapy.

### EXAMPLES

### Example 1

### I) Synthesis

### Reference being Made to FIG. 1

### 2′-cyano-8′-formyl-N-methyl-1,1a,5a,6-tetrahydroacrido-[4,5,5a,6-bcd]-annulated 2,3-dihydromesoporphyrin dimethylester nickel complex (compound 4)

### a) Preparation of the Vilsmeier Reagent:

POCl₃ (0.40 ml, 4.3 mmol) was added dropwise to N-methylformanilide (0.55 ml, 4.5 mmol) after which the mixture was stirred for 30 minutes at room temperature, resulting in the Vilsmeier reagent N-chloromethylene-N-methyl-N-phenyl ammonium dioxodichlorophosphate 3.

### b) Preparation of Compound 4:

The mixture obtained under a) was added to a stirred solution of 500 mg (0.71 mmol) of 5-(2′-cyanovinyl)mesoporphyrin dimethylester nickel complex 1 in 45 ml dichloroethane. Hence, there was a six-fold excess of Vilsmeier reagent. The reaction was followed with TLC using a mixture of 1% methanol in dichloromethane as the eluent. Starting compound 1 was prepared as disclosed in Eur. J. Org. Chem (2004) 19, pp. 4024-4038.

After one hour, 60 ml saturated aqueous sodium acetate was added to the reaction mixture until the pH=7, and the mixture was subsequently stirred overnight to hydrolyse the product mixture. The organic layer was separated, after which the solvent was evaporated under reduced pressure. N-methylformanilide and N-methylaniline in the crude product mixture were removed using silica gel flash chromatography with diethylether/hexanes (1:1). The porphyrin mixture was separated using silica gel chromatography with dichloromethane. The first brown fraction contained 160 mg (0.23 mmol, 28%) of the starting compound, the following green bands contained 105 mg (0.14 mmol, 20%) of a mixture of the nickel complexes of meso formylated 5-(2′-cyanovinyl) mesoporphyrin dimethylester. The last bright green band contained 130 mg (0.16 mmol, 22%) of the new compound 4 having a strong absorption at 650 nm.

### 2′-cyano-8′-formyl-N-methyl-1,1a,5a,6-tetrahydroacrido-[4,5,5a,6-bcd]-annulated 2,3-dihydromesoporphyrin dimethylester (compound 5)

50 mg (0.06 mmol) of compound 4 was dissolved in 4.0 ml of concentrated sulfuric acid and stirred at room temperature until the compound was dissolved completely (but not longer to prevent undesirable side reactions, in particular saponification of the cyano group). The reaction mixture was neutralized with an aqueous solution of sodium acetate and the precipitate was filtered off and washed with water. The crude product was purified with silica gel chromatography using a mixture of 1% methanol in dichloromethane as the eluent. The product was recrystallized from a mixture of dichloromethane and hexanes giving 29 mg (0.04 mmol, 62%) of compound 5.

### c) Spectroscopic Data of Compound 4:

Besides a strong absorption at 647 nm compound 4 also shows a strong peak at 2198 cm⁻¹ in the infrared spectrum indicating the presence of a nitrile function. Also peaks were present at 1676 cm⁻¹ and a Fermi doublet at 2730 and 2802 cm⁻¹, indicating that there is an aldehyde group attached to an electron-rich part of the molecule. HR-ESI mass spectroscopy gave an exact mass of m/z=847.3133, which corresponds with an elemental composition of ¹²C₄₈¹H₄₉¹⁴N₆¹⁶O₅⁵⁸Ni⁺ (calculated: m/z=847.3118) this corresponds with the monoprotonated form of compound 4. Removal of nickel with concentrated H₂SO₄ gives the nickel free derivative 5 of which the long-wavelength absorption has shifted to 687 nm (FIG. 1), which is very far into the red. Based on peaks in the IR-spectrum at 2194 cm⁻¹, 1674 cm⁻¹ and the Fermi doublet at 2720 and 2760 cm⁻¹, it is concluded that the nitrile function and the aldehyde function are still present. The HR ESI mass spectrum shows the parent peak at m/z=791.3931 which corresponds to an elemental composition of ¹²C₄₈¹H₅₁¹⁴N₆¹⁶O₅⁺ (calculated m/z=791.3921). This corresponds to the monoprotonated form of ¹²C₄₈¹H₅₀¹⁴N₆¹⁶O₅, this corresponds to the removal of one Ni²⁺ and the introduction of two hydrogen atoms. The unsaturation number is 27 (double bond equivalents; DBE). The elemental composition of 5-(2′-cyanovinyl) mesoporphyrin dimethylester is ¹²C₃₉¹H₄₃¹⁴N₅¹⁶O₄ with unsaturation number 21. This means that compound 5 contains 9 carbon atoms, 7 hydrogen atoms, one nitrogen atom and one oxygen atom more than 5-(2′-cyanovinyl)mesoporphyrin dimethylester. Also the unsaturation number in compound 5 has increased with 6 units. These facts indicate that the carbon, hydrogen and nitrogen atoms of reagent 3 have been included in the structure of compound 5 and that in addition an aldehyde group is present, presumably attached to the benzene ring of the N-methyl aniline group that now is incorporated in structure of compound 5. These data were corroborated by ¹H NMR and ¹³C NMR, which spectroscopic techniques allowed the present inventors to unequivocally establish and confirm the structure of compound 5.

The structure of compound 5 has three chiral carbon atoms (2, 3 and 2⁸). However due to the bridging aniline structure at 2 and 2⁸ the substituents on 2, 3, and 2⁸ are forced in a syn, syn orientation. This results in the fact that compound 5 occurs in only two enantiomeric forms. These two enantiomers have been fully separated with HPLC using a Daicel Chiralcel OD column (VWR, Amsterdam, the Netherlands) and a hexane/ethanol mixture (75/25 v/v) as the eluent. The CD-spectra of the eluting-peaks are exact mirror images of each other. Both enantiomers have electronic absorption spectra identical to that of compound 5. These results show that indeed compound 5 is a 1:1 mixture of two enantiomers, which are easily separated into the optically pure forms at a semi-preparative scale.

### II) Photochemical Singlet Oxygen Generation

Compound B dissolved in toluene was excited with a 9 mJ, 15 ns laser pulse at 532 nm from a home-built Q-switched frequency doubled Nd:YAG laser operating at 10 Hz. Emission from singlet oxygen at 1270 nm was detected at 90° to the quartz cuvette by an InGaAs photodiode, amplified and then signal-averaged (256 shots) on a digital oscilloscope (HP infinium). A 1260 nm interference filter (FWHM=75 nm) was used to protect the detector from laser light and sensitizer fluorescence. Typical decay times were 28±1 μs, which is characteristic for singlet oxygen decay in toluene (M. Okamoto et al, J. Phys. Chem., 1993, 97, pp. 177-180.) Tetraphenylporphyrin was used as a standard to determine the relative singlet oxygen yield (Φ_{Δ}) (F. Wilkinson, et al, J. Phys. Chem. Ref. Data., 1993, 22, pp. 113-2162.). The relative quantum yield for compound 5 in air saturated toluene was 0.72. No photo degradation as measured by changes in the absorption spectrum of compound 5, was observed under these conditions. This stability of the compound according to the invention as exemplified by compound 5 towards singlet oxygen is surprising. In many porphyrins with appending double bonds the double bond reacts chemically with the singlet oxygen generated by the system. E.g. the purpurin obtained by R. B. Woodward during the total synthesis of chlorophyll a is photo-oxygenated by the singlet oxygen generated by the purpurin derivative itself leading to cleavage of the double bond in the annulated cyclopentene ring (ref: R. B. Woodward et. al. J. Am. Chem. Soc. 1960, 82, p 3800-3802). In hindsight and without wishing to be bound by any particular theory, the stability of compound 5 towards singlet oxygen attack on the appending double bond could be explained by the low electron density of the double bond due to the electron withdrawing capacity of the nitrile function. The hydrogen atom attached to carbon-2⁸ is both allylic and geminal to a tertiary amine function which makes it in general very reactive towards singlet oxygen and other oxidizing agents. However due to structural restrictions caused by the presence of the bulky aromatic group (i.e. here the N-methyl aniline group) almost perpendicular to the plane of the porphyrin ring, the hydrogen at position 2⁸ is forced in the plane of the cyclohexadiene ring preventing any stabilization of the species that results from the removal of the 2⁸-hydrogen by the double bond and the aniline function. It is a fortunate coincidence that the precise structure resulting from the reaction performed according to the invention makes the compounds according to the invention stable towards attack by singlet oxygen. The nickel complex 4 was investigated in the same way; no singlet oxygen was observed (Φ_{Δ}<0.01), in accordance with the ordinary effect of nickel in porphyrin complexes.

### III) Biological Effects

First a toxicity test of compound 5 in cultures of A 549 lung carcinoma cells (American Type Culture Collection) was carried out in the dark. The cells were incubated in a medium containing compound 5 at various concentrations. The cultures were left overnight in the dark at 37° C. Using the MTT (3-(4,5-dimethylthiazol-2-yl)-2,5-diphenyltetrazolium bromide) assay based on the methods of Tada et al (J. Immunol. Meth., 1986, 93, pp. 157-165) and J. Carmichael et al (Cancer Res., 1987, 47, pp. 936-942.) the cell survival was obtained as a percentage of untreated cells of control (FIG. 2). Even at concentrations up to 30 μg/ml in the medium cell survival was complete (within the experimental error), showing that compound 5 is essentially not toxic towards the lung carcinoma cells in the dark. After incubating the lung cancer cells for 4 hours with compound 5 in the dark followed by illumination with white halogen light of 30 mW/cm² (270 kJ/m²) the lung carcinoma cells were killed for 70% at a concentration of 0.10 μg/ml, which is comparable to the results found for mTHPC (Foscan®) (Scotia, FIG. 3). This shows that in the presence of oxygen (from the ambient atmosphere) and light compound 5 acts as a very efficient photodynamic agent.

### Example 2

### 2′-aminocarbonyl-8′-formyl-N′-methyl-1′,1a′,5a′,6′-tetrahydroacrido[4,5,5a,6-bcd]-annulated 2,3-dihydromesoporphyrin dimethylester (compound 6)

### I) Synthesis

50 mg (0.06 mmole) of compound 5 was treated with 10 ml of concentrated sulfuric acid and stirred for 16 hours at 35-40° C. The reaction mixture was neutralized with a saturated aqueous sodium carbonate solution at 0° C. and then extracted with dichloromethane. The organic layer was dried with MgSO₄ and concentrated under reduced pressure. The crude mixture was purified by silica gel chromatography using a mixture of 5% methanol in dichloromethane as the eluent. The product was recrystallized from a mixture of methanol and hexanes giving 25 mg (0.03 mmole, 50%) of compound 6 (FIG. 4).

### II) Photosensitizer Activity

The oxygen consumption (which serves as an indirect measure for the photosensitizer's production capacity of singlet oxygen) was measured with an YSI (Yellow Springs Instrument, USA) model 5300 oxygen consumption meter equipped with a Clarke type electrode. The lamp used was a standard halogen lamp for a slide projector. The sample cuvettes were immersed in a thermostatic bath and histidine (2 mM in Phosphate Buffered Saline (PBS), pH 7.4) was chosen as a substrate for oxydation. The Clark electrode recorded the oxygen level in the solution during the illumination time. The production of reactive oxygen species and the subsequent reaction with the substrate leads to a decrease in the oxygen level of the PBS solution. The oxygen consumption rate was estimated by calculating the slope of the straight line which fits the beginning of the oxygen level decrease curve (for reference see: Damoiseau, X., Schuitmaker, J. J., Lagerberg, J. W. M., Hoebeke, M. (2001). Increase of the photosensitizing efficiency of Bacteriochlorine a by liposome incorporation, J. Photochem. Photobiol. B: Biol., 60, 50-60.). Compound 6 caused a decrease in dissolved oxygen of 34% per minute.

### Example 3

### 2′-cyano-8′-formyl-N′-methyl-1′,1a′,5a′,6′-tetrahydroacrido[4,5,5a,6-bcd]-annulated 2,3-dihydromesoporphyrin dicarboxylic acid (compound 7)

### I) Synthesis

To 250 mg (0.3 mmole) of compound 5 dissolved in 50 ml of pyridine 25 ml of a 1 N aqueous solution of sodium hydroxide was added and the mixture was stirred for 16 hours at room temperature. The solution was neutralized with 25 ml of a 1 N aqueous hydrochloric acid solution. Water was added and the water layer was extracted twice with dichloromethane. The organic layer was dried with MgSO₄, concentrated under reduced pressure and coevaporated once with toluene. The crude mixture was purified by silica gel chromatography using a mixture of 10% methanol in dichloromethane as the eluent. The product was recrystallized from a mixture of dichloromethane and hexanes giving 170 mg (0.2 mmole, 68%) of compound 7 (FIG. 4).

### II) Photosensitizer Activity

The oxygen consumption was determined as described in example 2, under II). Compound 7 showed a satisfactory value of 20% per minute.

### III) In Vivo Experiments

### a) AMD Model in Fertilized Chicken Eggs:

Age-related Macula Degeneration (AMD) is an eye affliction that eventually leads to blindness. For studies to treat this affliction, an AMD model exists, the so-called CAM (chorioallantoic membrane) model in fertilized chicken eggs. The blood vessels in this chorioallantoic membrane mimic a number of relevant features characteristic for the blood vessels in the eye involved in causing AMD. Experiments with compound 7 were performed at the department of Professor Hubert van den Bergh of the EPFL in Lausanne. Different doses of compound 7 (dissolved in Water:PEG-400:1-Methyl-2-pyrrolidone (50:30:20 v/v), pH 8.1) were injected in the CAM blood vessels and the vessels were illuminated after an 1 minute time-to-treat interval using different amounts of light energy from a far red light source. In the experiments, Visudyne™ (which has an absorption peak at 690 nm), the only photosensitizer approved for clinical use in AMD, was used as the control.

Compound 7 was as potent (already with a low light dose of 24 J/cm² and 1 mg/kg of body weight of photosensitizer 7 the desired grade 3 vessel damage could be obtained) as Visudyne™ in closing blood vessels in this model. FIG. 7 shows the closing of the small CAM blood vessels resulting from vessel damage obtained by Photo Dynamic Therapy (PDT) using 12 J/cm² at 1 mg/kg body weight of compound 7. (Legend: A: Compound 7 fluorescence angiography before PDT; B: compound 7 fluorescence angiography during PDT, shown in the circle is the red light irradiation area; C: sulforhodamine 101 fluorescence angiography 18 hours post-treatment).

However, and more importantly, compound 7 showed much less leakage from the CAM blood vessels than Visudyne™, as established by fluorescence microscopy (note the excellent contrast between vessels and background in pictures A and B in FIG. 7. Please note that the poor contrast as compared to compound 7 in picture C is due to leaking sulforhodamine 101 used for fluorescence angiography). The reduced leakage is an important observation, because it is, generally believed that part of the unwanted PDT damage that is observed in humans in the immediate surroundings of the Visudyne™ treated AMD blood vessels is caused by leakage of Visudyne™ from these abnormal blood vessels. This unwanted, leakage induced PDT damage caused by Visudyne™ results in more collateral damage in comparison with the situation where there is no leakage, which results in a limitation for therapy with this known compound. Finally, the above results is evidence that the compounds according to the present invention may be used in a pharmaceutical composition suitable for the treatment of vascular anomalies, for example naevus flammeus.

### b) Mouse Tumor Model:

Experiments were performed to investigate the suitability of compound 7 for the photodynamic therapy (PDT) treatment of tumors, in particular subcutaneous mammary CaNT tumors. These experiments were performed by Photopharmica Ltd. under the guidance of Professor Stan Brown in the Centre for Photobiology and Photodynamic Therapy (Leeds, UK). Different doses of compound 7 (dissolved in Water:PEG-400:1-Methyl-2-pyrrolidone (50:30:20 v/v), with a maximum concentration of 0.5 mg/ml at pH 8.1) were intravenously injected and tumors were illuminated with red light (685±15 nm, 60 J/cm², 50 mW/cm²) at different intervals after injection.

This study shows that compound 7 has good tumor killing potential in vivo at around 2.5 mg/kg and that this PDT effect is already present immediately (2 minutes!) after injection and virtually absent after 180 minutes. The potency of compound 7 in this model is estimated somewhere between the potency of the commercially available tumor photosensitizers Foscan (more potent than compound 7) and Photofrin™ (2.5 times less potent than compound 7 in this mouse model), with compound 7 having the advantage of lacking skin photosensitivity at the effective tumor dose within 24 hours after drug administration. In addition, compound 7 has the advantage that it can be activated by light in the far red (687 nm), which has a much better tissue penetration than the 628 nm light normally used for Photofrin™ and the 652 nm light used for Foscan™. FIG. 8 shows the percentage of necrotic area (% A) versus the dose D of compound 7 (designated PB109) in the figure. PB07 is a compound covered by a co-pending application of applicant and is of no importance here. Photofrin™ (designated as PHP in FIG. 8) clearly requires a higher dose for the same effect. FIG. 9 demonstrates that the powerful phototoxic effect of compound 7 (PB109) vanishes rapidly in a couple of hours, whereas Photofrin™ (PHP) remains phototoxic after several hours, that is, even after the treatment with light.

Damage to internal tissue surrounding the tumor was comparable to Photofrin™ in this model. It was observed that compound 7, in contrast to Photofrin™, did cause less damage to the skin overlaying the tumor after light treatment. This skin damage is part of the reason why light treatment can only be started 48 hours after Photofrin™ administration. A similar situation exists for Foscan™. Clinicians do not like this long time-to-treat interval, because (i) it is from a logistics point of view more difficult to organize, (ii) the procedure cannot be used during surgery as an immediate add-on therapy in case not all tumor can be surgically removed, and (iii) it is not a very efficient use of the clinician's time. In addition, the patients do not like it, because (iv) they need to come twice to the hospital and (v) during this waiting period, the patient needs to avoid contact with light.

From a clinical point of view the ideal photosensitizer should not only (i) be activated in the far red and has a (ii) very short drug-to-light interval in combination with (iii) no skin photosensitivity, but it should also have (iv) a good tumor selectivity. Although compound 7 seems to be much better regarding the first three characteristics, it appears to have a similar lack of tumor selectivity as Foscan™ and Photofrin™.

If the treatment with light is performed a couple of hours after injection of compound 7, there was no effect with respect to tumor-necrosis and skin damage, as discussed above. Without wishing to be bound by any particular theory, it is believed that this is caused by rapid clearance of compound 7 from the body, which is an unexpected but clear advantage.

Due to this, the patient can start his normal activities under normal light conditions almost immediately after treatment. With most other sensitizers, the patient needs to be protected from light for several weeks.

From the literature, it is known that photosensitizers that have been proven to work in one particular tumor model, also show activity against other tumors. Therefor it is believed that the photosensitizers according to the present invention will not only be useful for our tested mammary tumor, but also for the treatment of other primary tumors and/or metastases thereof.

### Example 4

### 2′-cyano-9′,N′-dimethyl-8′-formyl-1′,1a′,5a′,6′-tetrahydroacrido[4,5,5a,6-bcd]-annulated 2,3-dihydromesoporphyrin dimethylester nickel complex (compound 6a), and 2′-cyano-7′,N′-dimethyl-8′-formyl-1′,1a′,5a′,6′-tetrahydroacrido[4,5,5a,6-bcd]-annulated 2,3-dihydromesoporphyrin dimethylester nickel complex (compound 8b)

### a) Preparation of the Vilsmeier Reagent:

25 grams (0.2 mole) of N-methyl-m-toluidine was dissolved in 32.5 grams (0.3 mole) trimethylorthoformate. After addition of 0.25 ml of concentrated sulfuric acid the solution was refluxed for half an hour. The excess trimethylorthoformate and the liberated methanol were distilled off at atmospheric pressure. The residue was fractionally distilled at reduced pressure. The pure N-methyl-m-methylformanilide was collected as a slightly yellow oil at 110-120° C. (0.15 mm Hg). The yield was 6.4 grams (0.04 mole, 20%).

Using the N-methyl-m-methylformanilide of the previous step as the starting compound, the Vilsmeier reagent N-chloromethylene-N-methyl-methylphenyl ammonium dioxodichlorophosphate (3a) was prepared in the same way and at the same scale as described for compound 4 (see example 1).

### b) The Vilsmeier Reaction:

The Vilsmeier reaction was performed in a similar way as described for the synthesis of compound 4, using 500 mg of compound 1. The yield of the starting material after purification was: 120 mg (0.17 mmole, 21), the formylated byproduct (compound 16 in FIG. 1): 100 mg (0.13 mmole, 19%) and of the (1:1) mixture of compounds 8a and 8b: 130 mg (0.16 mmole, 22%) (FIG. 5).

### Example 5

### 2′-cyano-9′,N′-dimethyl-8′-formyl-1′,1a′,5a′,6′-tetrahydroacrido[4,5,5a,6-bcd]-annulated 2,3-dihydromesoporphyrin dimethylester (compound 9)

130 mg (0.16 mmole) of the mixture of compounds 8a and 8b was converted into 75 mg (0.1 mmole, 60%) of compound 9 (+ its isomer) as described for the conversion of compound 4 into compound 5. 14 mg of the pure isomer 9 was obtained after two recrystallizations from a mixture of dichloromethane and hexanes (FIG. 5).

### Example 6

### 2′-cyano-8′-formyl-N′-methyl-1′,1a′,5a′,6′-tetrahydroacrido[4,5,5a,6-bcd]-annulated octaethyl porphyrin nickel complex (10) and 2′-cyano-8′-formyl-N′-methyl-1′,1a′,5a′,6′-tetrahydroacrido[4,5,5a,6-bcd]-annulated octaethyl porphyrin (compound 11).

### I) Synthesis

### a) Preparation of the Vilsmeier Reagent:

POCl₃ (1.2 ml, 13 mmol) was added dropwise to N-methylformanilide (1.65 ml, 13.5 mmol) after which the mixture was stirred for 30 minutes at room temperature, resulting in Vilsmeier reagent N-chloromethylene-N-methyl-N-phenyl ammonium dioxodichloro phosphate (3).

### b) The Vilsmeier Reaction:

This mixture obtained was added to a stirred solution of 600 mg (1 mmol) of nickel octaethyl porphyrin (1a) (FIG. 6) in 65 ml dichloroethane. Hence, there was a thirteen-fold excess of Vilsmeier reagent. The reaction was followed with TLC using dichloromethane as the eluent. After one hour the reaction was complete and the solvent was removed under reduced pressure. The residue was taken up in 70 ml methanol and added to 85 ml of a saturated aqueous solution of sodium acetate. The mixture was stirred overnight to hydrolyze the product. The organic layer was separated after which the solvent was evaporated under reduced pressure. Purification with silicagel chromatography with dichloromethane as the eluent gives 625 mg (1 mmol, 100%) of 5-formyl nickel octaethyl porphyrin (1b) (FIG. 6).

### c) The Horner-Emmons Reaction:

Sodium hydride (60% in mineral oil) (500 mg, 12.5 mmole) was washed three times with hexane after which 50 ml tetrahydrofuran and diethylphosphono acetonitril (2.6 g, 14.7 mmole) were added. The mixture was stirred at room temperature for 15 minutes until the sodium hydride was dissolved completely, after which a solution of 625 mg (1 mmole) of compound 1b in THF (50 ml) was added. After 12 hours stirring at room temperature the reaction was complete as observed by TLC, water was added and the mixture was extracted three times with dichloromethane. The organic layer was dried with MgSO₄ and concentrated under reduced pressure. The product was purified with silicagel chromatography using dichloromethane as the eluent, giving 650 mg (1 mmole, 100%) of 5-(2′-cyanovinyl) octaethyl porphyrin nickel complex (2) (FIG. 6).

### d) The Annulation Reaction:

Compound 10 was synthesized in a similar way as described for the conversion of compound 1 into compound 4 (example 1). The product was purified by silica gel chromatography using a mixture of ether and hexanes (v/v=1/1) as the eluent. Starting from 650 mg, (1 mmole) of compound 2, the yield of the starting material after purification was: 120 mg (0.17 mmole, 21%), of the formylated byproduct: 100 mg (0.13 mmole, 19%) and of compound 10: 180 mg (0.27 mmole, 27%).

### e) Preparation of Compound 11:

120 mg (0.18 mmole) of compound 10 was converted into 80 mg (0.12 mmole, 66%) of compound 11 (FIG. 6) as described for the conversion of compound 4 to compound 5 (example 1). The product was purified by silica gel chromatography using a mixture of ether and hexanes (v/v=1/1) as the eluent. The product was recrystallized from ether and hexanes.

### II) Photosensitizer Activity

The photosensitizer activity was measured as disclosed in Example 2 under II). Compound 11 caused a decrease in dissolved oxygen per minute of 50%.

1. A method of preparing a porphyrin derivative from a meso-acrylonitrile-substituted porphyrin compound, wherein said meso-acrylonitrile-substituted porphyrin compound contains a coordinated bivalent metal ion in the porphyrin macrocycle, characterized in that the meso-acrylonitrile-substituted porphyrin compound, in a form wherein said meso acrylonitrile-substituted porphyrin compound is completed with a bivalent metal ion, is contacted with a Vilsmeier reagent having a reactive motif 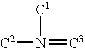
containing a quaternary nitrogen atom which is directly linked to two carbon atoms C¹, C² wherein at least one of said carbon atoms is part of an aromatic moiety, and wherein said quaternary nitrogen atom is directly linked to a carbon atom C³ via a double bond, said carbon atom C³ carrying a halogen atom chosen from fluoro, chloro, bromo and iodo,
with the restriction that at least one C atom of the aromatic moiety ortho with respect to the quaternary nitrogen is unsubstituted;
to convert said meso-acrylonitrile-substituted porphyrin compound into a porphyrin derivative having the ring system of the Vilsmeier reagent condensed to its porphyrin macrocycle via two new 6 membered rings.

2. Method according to claim 1, characterized in that a Vilsmeier reagent is used chosen from the group consisting of 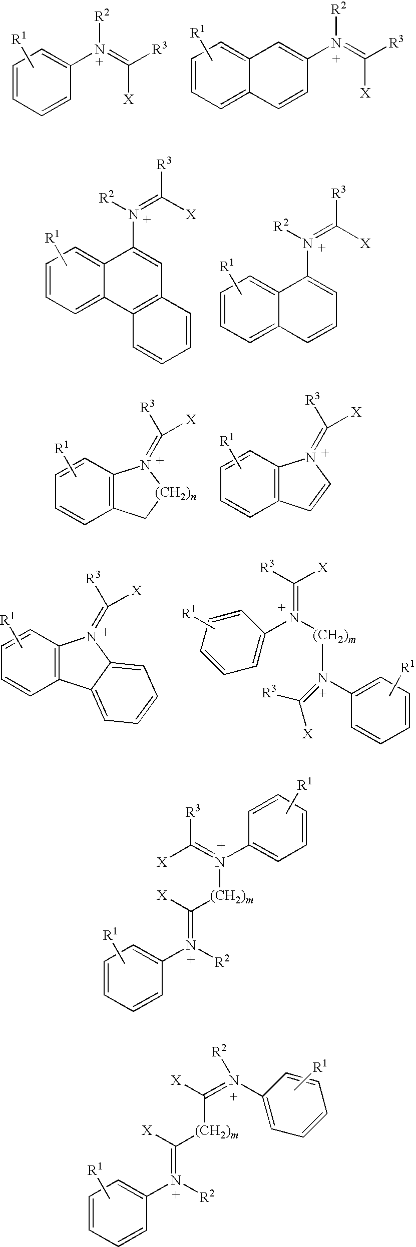
wherein
n is an integer from 0 to 3
m is an integer from 1 to 8
X is an halogen atom chosen from fluoro, chloro, bromo, and iodo;
R¹ is
hydrogen, or
has the same meaning as defined for R², in which case it may be condensed with an aromatic ring of the ring system of the Vilsmeier reagent, whose aromatic ring carries the quaternary nitrogen atom;
R² is
a C₁₋₆ alkyl residue, wherein said alkyl residue may optionally be substituted with one or more substituents independently chosen from the group of linear or branched C₁₋₆ alkoxy, linear or branched C₁₋₆ alkylthio, linear or branched C₂₋₆ alkenyl, linear or branched C₂₋₆ alkynyl, chloro, bromo or fluoro, carbonyl, C₆₋₁₂ aryl, or amino substituted with two substituents independently chosen from the group of linear or branched C₁₋₆ alkoxy, linear or branched C₁₋₆ alkylthio, and C₆₋₁₂ aryl where these substituents of the amine group may optionally be substituted with fluoro, chloro, bromo, and iodo; wherein the saturated or unsaturated C₁₋₆ alkyl residue may be part of a ring system condensed with the aromatic ring carrying the nitrogen atom of the Vilsmeier reagent;
or
a C₆₋₂₂ aryl residue, wherein said aryl residue may optionally be substituted with one or more substituents independently chosen from the group of linear or branched C₁₋₆ alkyl, linear or branched C₁₋₆ alkoxy, linear or branched C₁₋₆ alkylthio, linear or branched C₂₋₆ alkenyl, linear or branched C₂₋₆ alkynyl, chloro, bromo or fluoro, carbonyl, or amino substituted with two substituents independently chosen from the group of linear or branched C₁₋₆ alkoxy, linear or branched C₁₋₆ alkylthio, and C₆₋₁₂ aryl where these substituents of the amine group may optionally be substituted with fluoro, chloro, bromo, and iodo; wherein the C₆₋₁₂ aryl residue may be part of a ring system condensed with the aromatic ring carrying the nitrogen atom of the Vilsmeier reagent; and
R³ is hydrogen or is as defined for R².

3. Method according to claim 1 or 2, characterized in that the halogen atom is chloro.

4. Method according to any of the preceding claims, characterized in that a meso-acrylonitrile-substituted porphyrin compound of the formula (I) is used as a starting compound 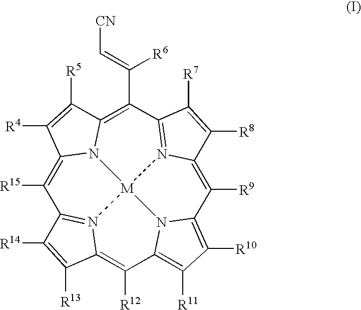
wherein
R⁴, R⁵, R⁷ and R⁸ represent, independently of each other, hydrogen, linear or branched (C₁₋₈) alkyl, or linear or branched (C₁₋₈)alkyl C(O)O(C₁₋₈)alkyl, wherein the alkyl groups may optionally be substituted with fluoro, chloro, bromo, iodo, nitrile, (C₁₋₈) thioether, and (C₁₋₈) alkoxy;
R⁶ represents hydrogen, nitrile, monocyclic, bicyclic or tricyclic (C₆₋₁₄) aryl, or (C₁₋₄) alkyl wherein the aryl; and alkyl group may optionally be substituted with fluoro, chloro, bromo, iodo, nitrile, (C₁₋₈) thioether, and (C₁₋₈) alkoxy, and the alkyl group may be substituted with a monocyclic, bicyclic or tricyclic (C₆₋₁₄) aryl;
R⁹ to R¹⁵ represent independently of each other, hydrogen, linear or branched (C₁₋₈) alkyl, linear or branched (C₁₋₈)alkyl C(O)O(C₁₋₈)alkyl, wherein n is an integer of 0 to 4, CH₂═CH—, a monocyclic, bicyclic or tricyclic (C₃-C₁₄) aryl, said aryl optionally containing one or more nitrogen atoms as heteroatoms, and R⁹, R¹², and R¹⁵ may in addition represent an acrylonitrile group substituted with R⁶′, wherein R⁶′ is as defined for R⁶;
and
M represents a bivalent metal ion or two hydrogen atoms.

5. Method according to any of the preceding claims, characterized in that the porphyrin derivative is formylated.

6. Method according to any of the preceding, claims, characterized in that the bivalent metal ion is removed or replaced by another metal ion.

7. Method according to any of the preceding claims, characterized in that the nitrogen atom of the ring system is quaternized.

8. Method according to any of the preceding claims, characterized in that the meso-acrylonitrile-substituted porphyrin compound is derived from a precursor porphyrin compound chosen from the group of i) hemin, and ii) heme.

9. A porphyrin derivative obtainable with the method according to any of the claims 1 to 8, its enantiomers, saponified esters thereof as well as the addition salts thereof with an acid or base.

10. A porphyrin derivative chosen from the group consisting of
2′-Cyano-8′-formyl-N-methyl-1′,1a′,5a′,6′-tetrahydroacrido[4,5,5a,6-bcd]-annulated 2,3-dihydromesoporphyrin dimethylester;
2′-Cyano-8′-formyl-N-methyl-1′,1a′,5a′,6′-tetrahydroacrido[4,5,5a,6-abt]-annulated 2,3-dihydromesoporphyrin dimethylester;
2′-Cyano-8′-formyl-N-methyl-1′,1a′,5a′,6′-tetrahydroacrido[4,5,5a,6-efg]-annulated 7,8-dihydromesoporphyrin dimethylester;
2′-Cyano-8′-formyl-N-methyl-1′,1a′,5a′,6′-tetrahydroacrido[4,5,5a,6-ghi]-annulated 7,8-dihydromesoporphyrin dimethylester;
2′-Cyano-8′-formyl-N-methyl-1′,1a′,5a′,6′-tetrahydroacrido[4,5,5a,6-jkl]-annulated 12,13-dihydromesoporphyrin dimethylester;
2′-Cyano-8′-formyl-N-methyl-1′,1a′,5a′,6′-tetrahydroacrido[4,5,5a,6-lmn]-annulated 12,13-dihydromesoporphyrin dimethylester;
2′-Cyano-8′-formyl-N-methyl-1′,1a′,5a′,6′-tetrahydroacrido[4,5,5a,6-opq]-annulated 17,18-dihydromesoporphyrin dimethylester;
2′-Cyano-8′-formyl-N-methyl-1′,1a′,5a′,6′-tetrahydroacrido[4,5,5a,6-qrs]-annulated 17,18-dihydromesoporphyrin dimethylester;
2′-Cyano-8′-formyl-N-methyl-1′,1a′,5a′,6′-tetrahydroacrido[4,5,5a,6-bcd]-annulated 2,3-dihydromesoporphyrin;
2′-Cyano-8′-formyl-N-methyl-1′,1a′,5a′,6′-tetrahydroacrido[4,5,5a,6-abt]-annulated 2,3-dihydromesoporphyrin;
2′-Cyano-8′-formyl-N-methyl-1′,1a′,5a′,6′-tetrahydroacrido[4,5,5a,6-efg]-annulated 7,8-dihydromesoporphyrin;
2′-Cyano-8′-formyl-N-methyl-1′,1a′,5a′,6′-tetrahydroacrido[4,5,5a,6-ghi]-annulated 7,8-dihydromesoporphyrin;
2′-Cyano-8′-formyl-N-methyl-1′,1a′,5a′,6′-tetrahydroacrido[4,5,5a,6-jkl]-annulated 12,13-dihydromesoporphyrin;
2′-Cyano-8′-formyl-N-methyl-1′,1a′,5a′,6′-tetrahydroacrido[4,5,5a,6-lmn]-annulated 12,13-dihydromesoporphyrin;
2′-Cyano-8′-formyl-N-methyl-1′,1a′,5a′,6′-tetrahydroacrido[4,5,5a,6-opq]-annulated 17,18-dihydromesoporphyrin,
2′-Cyano-8′-formyl-N-methyl-1′,1a′,5a′,6′-tetrahydroacrido[4,5,5a,6-qrs]-annulated 17,18-dihydromesoporphyrin;
2′-Aminocarbonyl-8′-formyl-N-methyl-1′,1a′,5a′,6′-tetrahydroacrido[4,5,5a, 6-bcd]-annulated 2,3-dihydromesoporphyrin dimethylester;
2′-Aminocarbonyl-8′-formyl-N-methyl-1′,1a′,5a′,6′-tetrahydroacrido[4,5,5a,6-abt]-annulated 2,3-dihydromesoporphyrin dimethylester;
2′-Aminocarbonyl-8′-formyl-N-methyl-1′,1a′,5a′,6′-tetrahydroacrido[4,5,5a,6-efg]-annulated 7,8-dihydromesoporphyrin dimethylester;
2′-Aminocarbonyl-8′-formyl-N-methyl-1′,1a′,5a′,6′-tetrahydroacrido[4,5,5a,6-ghi]-annulated 7,8-dihydromesoporphyrin dimethylester;
2′-Aminocarbonyl-8′-formyl-N-methyl-1′,1a′,5a′,6′-tetrahydroacrido[4,5,5a,6-jkl]-annulated 12,13-dihydromesoporphyrin dimethylester;
2′-Aminocarbonyl-8′-formyl-N-methyl-1′,1a′,5a′,6′-tetrahydroacrido[4,5,5a,6-lmn]-annulated 12,13-dihydromesoporphyrin dimethylester;
2′-Aminocarbonyl-8′-formyl-N-methyl-1′,1a′,5a′,6′-tetrahydroacrido[4,5,5a,6-opq]-annulated 17,18-dihydromesoporphyrin dimethylester;
2′-Aminocarbonyl-8′-formyl-N-methyl-1′,1a′,5a′,6′-tetrahydroacrido[4,5,5a,6-qrs]-annulated 17,18-dihydromesoporphyrin dimethylester
2′-Aminocarbonyl-8′-formyl-N-methyl-1′,1a′,5a′,6′-tetrahydroacrido[4,5,5a,6-bcd]-annulated 2,3-dihydromesoporphyrin;
2′-Aminocarbonyl-8′-formyl-N-methyl-1′,1a′,5a′,6′-tetrahydroacrido[4,5,5a,6-abt]-annulated 2,3-dihydromesoporphyrin;
2′-Aminocarbonyl-8′-formyl-N-methyl-1′,1a′,5a′,6′-tetrahydroacrido[4,5,5a,6-efg]-annulated 7,8-dihydromesoporphyrin;
2′-Aminocarbonyl-8′-formyl-N-methyl-1′,1a′,5a′,6′-tetrahydroacrido[4,5,5a,6-ghi]-annulated 7,8-dihydromesoporphyrin;
2′-Aminocarbonyl-8′-formyl-N-methyl-1′,1a′,5a′,6′-tetrahydroacrido[4,5,5a,6-jkl]-annulated 12,13-dihydromesoporphyrin;
2′-Aminocarbonyl-8′-formyl-N′-methyl-1′,1a′,5a′,6′-tetrahydroacrido[4,5,5a,6-lmn]-annulated 12,13-dihydromesoporphyrin;
2′-Aminocarbonyl-8′-formyl-N-methyl-1′,1a′,5a′,6′-tetrahydroacrido[4,5,5a,6-opq]-annulated 17,18-dihydromesoporphyrin;
2′-Aminocarbonyl-8′-formyl-N-methyl-1′,1a′,5a′,6′-tetrahydroacrido[4,5,5a,6-qrs]-annulated 17,18-dihydromesoporphyrin;
2′-aminocarbonyl-8′-formyl-N′-methyl-1′,1a′,5a′,6′-tetrahydroacrido[4,5,5a,6-bcd]-annulated 2,3-dihydromesoporphyrin dimethylester;
2′-cyano-8′-formyl-N′-methyl-1′,1a′,5a′,6′-tetrahydroacrido[4,5,5a,6-bcd]-annulated 2,3-dihydromesoporphyrin dicarboxylic acid;
2′-cyano-9′,N′-dimethyl-8′-formyl-1′,1a′,5a′,6′-tetrahydroacrido[4,5,5a,6-bcd]-annulated 2,3-dihydromesoporphyrin dimethylester; and
2′-cyano-8′-formyl-N′-methyl-1′,1a′,5a′,6′-tetrahydroacrido[4,5,5a,6-bcd]-annulated octaethyl porphyrin
as well as the addition salts thereof with pharmaceutically acceptable acid or base.

11. Use of a porphyrin derivative according to claim 9 or 10 for the preparation of a pharmaceutical composition for prevention of and/or treating benign, malignant, inflamed and infectious
1) skin and mucosa disorders;
2) vascular disorders;
3) tumors and pre-cancerous lesions;
4) opthalmology disorders;
5) gynaecological or urological disorders;
6) immunological disorders;
7) oral cavity or nasopharyngeal disorders.

12. Use according to claim 11, wherein the disorder is a vascular anomaly.

13. Use according to claim 12, wherein the vascular anomaly is Age-related Macula Degeneration.

14. Use according to claim 11, wherein the disorder is a primary tumor and/or metastases thereof.

15. Use according to claim 14, wherein the primary tumor and/or metastases thereof is a mammary tumor and/or metastases thereof.

16. Use of a porphyrin derivative according to claim 9 or 10 for the preparation of a composition
1) for photodetection of malignant and pre-malignant lesions;
2) for decontamination or pathogen reduction of liquids such biological fluids and contaminated water;
3) for decontamination or pathogen reduction of surfaces;
4) for use as insecticide.

17. A pharmaceutical composition comprising a porphyrin derivative according to claim 9 or 10 together with a pharmaceutically acceptable carrier or excipient.

18. The pharmaceutical composition according to claim 17, suitable for the treatment of benign, malignant, inflamed and infectious
1) skin and mucosa disorders;
2) vascular disorders;
3) tumors and pre-cancerous lesions;
4) opthalmology disorders;
5) gynaecological or urological disorders;
6) immunological disorders;
7) oral cavity or nasopharyngeal disorders.

19. The pharmaceutical composition according to claim 17 or 18, suitable for the treatment of a vascular anomaly.

20. The pharmaceutical composition according to claim 19, suitable for the treatment of Age-related Macula Degeneration.

21. A pharmaceutical composition according to claim 17 or 18, suitable for the treatment of a primary tumor and/or metastases thereof.

22. A pharmaceutical composition according to claim 21, suitable for the treatment of a mammary tumor and/or metastases thereof.
